(19) United States
(12) Patent Application Publication
(10) Pub. No.: US 2012/0116303 A1
(43) Pub. Date: May 10, 2012

(54) Kit for Providing an Artificial Stomach Entrance

(76) Inventor: Karl-Heinz Marx, Buchholz (DE)

(21) Appl. No.: 13/259,774

(22) PCT Filed: Mar. 25, 2010
(86) PCT No.: PCT/EP2010/001875

§ 371 Date: Nov. 15, 2011

(30) Foreign Application Priority Data
Mar. 25, 2009 (DE) 20 2009 004 536.6

(51) Int. Cl. A61B 17/34; A61M 25/04

(52) U.S. Cl. 604/98.01; 604/96.01; 604/164.01; 604/167.03; 604/167.04; 604/103.11

(57) ABSTRACT

Kit for providing an artificial stomach entrance, comprising a trocar with a trocar sleeve for penetrating the abdominal wall into the stomach and a PEG tube with a distal extension insertable into the stomach in the form of a balloon made of a flexible material if need be with slight stretchability, which has at least one opening and a proximally connecting tubular area, wherein a feeding channel extends from a proximal feeding opening of the tubular area through the tubular area up to the at least one opening and the balloon is connected with a fill channel, which extends along the tubular area up to the proximal end and has a fill opening with a seal.

### CROSS-REFERENCE TO RELATED APPLICATIONS

Not applicable.

### STATEMENT REGARDING FEDERALLY SPONSORED RESEARCH

Not applicable.

### BACKGROUND OF THE INVENTION

The invention relates to a kit for providing an artificial stomach entrance.

Implants or respectively PEG tubes for use in the percutaneous food supply are used in intragastral long- and short-term feeding in patients. This hereby concerns a feeding type, which is always practical when food intake via mouth and esophagus no longer functions. Percutaneous food supply is advantageous with respect to venous feeding in that the digestive tract is loaded with food.

For percutaneous food supply, a PEG tube is inserted into a puncture channel through the abdominal wall to the stomach and liquid food is then fed through the implant.

Currently in approximately 90% of the cases, the pull method according to Keimling is used to place the PEG tube. In this method, a gastroscope is inserted through the esophagus, with which the stomach is filled through air insufflation and thus pressed tightly against the abdominal wall. A light point in an outlet opening of the gastroscope can be observed in a dark space through the stomach or abdominal wall (diaphanoscopy). A cannula or respectively a tube with a needle is applied to the light point and the needle is pushed through. A thread is pushed through the cannula. The entire process is monitored via a video monitor. The picture from the inside of the stomach is transmitted to the outside via a video gastroscope, which is connected to a video process and this in turn to a monitor. A forceps is inserted through the gastroscope and the thread through the gastroscope is pulled out of the mouth of the patient. After this, the gastroscope is removed. When the gastroscope is no longer lying, the thread with the PEG tube is pulled into the stomach through the mouth of the patient and the PEG tube is pulled out of the abdominal wall on an appendage piece. Finally, a cover or respectively a seal is placed from the outside.

For one, the thread pull method is disadvantageous due to the high effort involved. Furthermore, the guiding of the PEG tube through the unsterile areas of the mouth and the subsequent insertion into the open wound in the abdominal wall area can lead to infections. In the literature, the most frequent postoperative complication is specified as peristomal wound infection, which occurs in up to 30% of cases using the thread pull method despite prophylactic antibiotic administration. A second gastroscopy may also be needed to check the position of the PEG tube, wherein the patient impact from the gastroscopies is very high.

After the puncture channel has healed, the PEG tube can be replaced by a so-called “button.” For the removal of the PEG tube, the gastroscope must be used again and the forceps must be inserted. The PEG tube can then be pulled out of the inside of the stomach by means of the forceps. Since the thread pull method can only be performed in the clinic or in practices performing endoscopies, patients must be moved there, which is also complicates use.

In a further developed method, a trocar with a trocar sleeve is applied from the outside to the abdominal wall and inserted during the diaphanoscopy. A PEG tube is inserted through the trocar sleeve. The insertion of the PEG tube is inspected optically from the stomach through a gastroscope. When the tube is inserted, a balloon is inflated on the end of the tube or respectively filled with liquid. The longitudinally separable cannula (splitting cannula) is then pulled apart or respectively separated and removed so that the PEG tube lies. Finally, a closure applied to the PEG tube is lowered onto the abdominal wall and a supply tube is placed onto the closure. This method only relates to the PEG tube (initial insertion). The button (second insertion) is placed after healing of the puncture channel in the previous manner.

PEG tubes and buttons of the aforementioned type are described in EP 0 667 763 B1.

In the case of the known trocars with trocar sleeve, the penetration of the abdominal wall and stomach is very laborious. There is a risk that the opposite-lying stomach wall will be damaged during penetration. Furthermore, the trocar pushes the stomach away.

Based on this, an object of the invention is to provide an easier-to-use kit for providing an artificial stomach entrance.

### BRIEF SUMMARY OF THE INVENTION

The kit according to the invention for providing an artificial stomach entrance comprises a trocar with a trocar sleeve for penetrating the abdominal wall into the stomach and a PEG tube with a distal extension insertable into the stomach in the form of a balloon made of a flexible material if need be with slight stretchability, which has at least one opening and a proximally connecting tubular area, wherein a feeding channel extends from a proximal feeding opening of the tubular area through the tubular area up to the at least one opening and the balloon is connected with a fill channel, which extends along the tubular area up to the proximal end and has a fill opening with a seal.

In the case of conventional PEG tubes, the balloon is made of an elastic material. During the filling of the balloon with a fluid, the material of the balloon is considerably stretched. Also, the balloon, which is supported on the interior wall of the stomach, is under constant pressure. This can lead to the balloon popping and the PEG tube no longer being supported on the inside of the stomach wall. However, in the case of the invention, the balloon is made of a flexible material, which is not stretchable or is slightly stretchable, if need be. The advantage of this balloon is that it is under less pressure than a conventional balloon after being filled with a fluid. As a result, the balloon of the PEG tube of the kit according to the invention is harder to damage.

The material of the balloon has less stretchability than silicon, which is used for conventional PEG tubes. In accordance with a preferred embodiment, the material for the balloon is PUR (polyurethane) or another flexible material with a similarly low stretchability. A plastic with a similarly low stretchability is preferably used.

In the case of a conventional PEG tube, the elastic balloon can mainly lie flat against the PEG tube. In the case of the PEG tube of the kit according to the invention, this is not generally the case since the balloon does not constrict elastically. In accordance with a preferred embodiment, the balloon lies in the collapsed state on the tubular area of the PEG tube and is secured in this state on the tubular area through securing means. The securing means prevents the balloon from opening before being pushed through the trocar sleeve into the stomach. The securing means protects the balloon from damage during insertion into the trocar sleeve. The securing means can for example be a solid medium applied to the balloon, which dissolves in the stomach of the patient through the effects of stomach acid. In the field of pharmaceuticals, gels are for example known, which are solid outside of the stomach and dissolve in the environment of the stomach and are biocompatible. Such gels can be used for example as securing means. The medium can contain an antiseptic substance. However, the securing means can also be formed by an insertion sleeve, into which the PEG tube is inserted before insertion into the trocar sleeve. With the help of the insertion sleeve, the PEG tube is inserted into the trocar sleeve and the insertion sleeve can then be removed from the PEG tube. This can happen before or after the PEG tube with the collapsed balloon is pushed out of the distal end of the trocar sleeve.

The kit for providing an artificial stomach entrance comprises a trocar with trocar sleeve for penetration through the abdominal wall into the stomach and a PEG tube insertable into it after removal of the trocar from the trocar sleeve, wherein the trocar has a trocar tip with a point angle of maximum 30°.

The kit according to the invention has a trocar with a trocar tip tapered in a previously unknown manner. It has a point angle of maximum 30°. The point angle is an angle between a surface line of the trocar tip and its center axis. The trocar tip is e.g. designed as a multi-edge tip and/or as a conical tip. In the case of the multi-edge tip, the point angle is the polished angle and, in the case of the conical tip, the point angle is half the conical angle. The particularly tapered trocar tip benefits a gradual penetration of the abdominal wall and stomach wall. A pressing on the stomach from inside by means of a gastroscope or a sewing of the stomach onto the abdominal wall can be foregone. It is sufficient to blow up the stomach with air via the gastroscope so that it is pushed from the inside onto the abdominal wall. The gastroscope for inserting air into the stomach can be used in addition to the observation of the piercing process. In contrast, the known trocars with a comparatively blunt tip first penetrate the abdominal wall upon application of high pressure, push into the stomach wall and pierce it abruptly.

In accordance with one embodiment, the point angle is maximum 20°. In accordance with another embodiment, the point angle is 5° to 12°.

In accordance with one embodiment, the trocar tip has a length of approx. 8 mm to 22 mm. For children, the length is preferably up to approx. 12 mm. For adults, it is preferably up to approx. 20 mm. Injuries during insertion into the stomach should be avoided through the restriction of the length. In accordance with one embodiment, the trocar has a diameter in the range of approx. 4 through 8 mm. A smaller diameter of up to approx. 4 mm is selected instead for children. For adults, the diameter can also be larger, namely up to approx. 8 mm.

In accordance with another embodiment, the trocar tip has a boring tip distally. The boring tip serves to bore a hole in the abdominal wall. For this, it preferably has a greater point angle than the trocar tip. The point angle of the boring tip is preferably 20°. Furthermore, it is preferably above 30°. The boring tip can be designed in particular as a multi-edge tip or as a conical tip.

The entire trocar tip is preferably conical. The trocar tip can be formed in particular from one single cone or from different conical sections. The distal, conical section can be a boring tip in particular. However, in the case of a generally conical boring tip, a multi-edge tip can also be present distally as a boring tip.

In accordance with one embodiment, trocar is polished in a sharp-edged manner. The gradual piercing and penetration of the abdominal wall and stomach wall can hereby be further facilitated.

In accordance with another embodiment, the trocar has a triangular tip.

In accordance with another embodiment, the conical tip is provided with an external thread. Due to the external thread, the gradual piercing can be supported through a screwing movement of the trocar. The conical tip can hereby be designed in a corkscrew-like manner

The kit for providing an artificial stomach entrance comprises a trocar with a trocar sleeve for penetrating the abdominal wall into the stomach and a PEG tube insertable into it after removal of the trocar from the trocar sleeve, wherein the trocar sleeve has a valve proximally, through which the trocar can be inserted into the trocar sleeve and which seals airtight after removal of the trocar from the trocar sleeve.

In the case of the kit according to the invention, the trocar with the trocar sleeve can pierce the abdominal wall and the stomach wall. It can hereby be ensured through the introduction of air into the stomach that the stomach lies against the stomach wall on the inside. When the trocar is removed from the trocar sleeve, excess pressure can escape from the stomach in the case of conventional kits. As a result, the stomach can detach from the abdominal wall and slide off the trocar sleeve so that the correct placement of the PEG tube with the distal end cannot be guaranteed in the stomach. In the case of the kit according to the invention, the proximal opening of the trocar sleeve can be sealed by the valve when the trocar is removed. As a result, the excess pressure remains in the stomach and the stomach wall does not slide off the trocar sleeve. As a result, the placement of the PEG tube is supported and simplified by the kit according to the invention.

The airtight sealing first guarantees that the air pressure in the stomach does not fall too much up to insertion of the PEG tube so that the stomach wall detaches to a considerable degree from the abdominal wall and slides off the trocar sleeve. Absolute air tightness is generally not required. However, it is sought to design the valve such that it is as airtight as possible after removal of the trocar.

The valve can generally be connected inseparably with the trocar sleeve. However, in accordance with a preferred embodiment, the trocar sleeve and the valve have holding means for detachably holding the valve on the trocar sleeve. This embodiment favors in particular a separate production of trocar sleeve and valve, since the valve can be mounted on the trocar sleeve later. Furthermore, in the case of this embodiment, the valve can be removable after use and replaceable with an unused valve. This is particularly advantageous when a reusable trocar sleeve is used. However, the removability of the valve can also be advantageous for the removal of the trocar sleeve from the insertion channel. This is the case in particular when the trocar sleeve can be broken down into two halves in the longitudinal direction in order to remove it from a PEG tube with a proximal broadening. The valve can first be removed, which facilitates the breaking down of the trocar sleeve into two halves.

In accordance with another embodiment, the valve and the trocar sleeve have snap-on means for snapping the valve to the trocar sleeve. As a result, the valve is particularly easily mountable and removable from the trocar sleeve.

The valve can be designed in a different manner. In accordance with one embodiment, it comprises a cover plate made of an elastic material or a material with a memory effect, which is affixed to the trocar sleeve on the edge and blocks the passage channel of the trocar sleeve and through which the trocar is piercable for insertion into the passage channel, wherein the penetration point in the cover plate closes after removal of the trocar due to the elastic reset forces or the memory effect. The cover plate lies elastically or due to the memory effect on the perimeter of the trocar during penetration of the trocar and the penetration point automatically closes when the trocar is removed. Sufficient sealing of the trocar sleeve is thereby achieved so that the excess pressure in the stomach does not drop to a detrimental degree up to placement of the PEG tube.

In accordance with another embodiment, the cover plate has an outwardly bent, circumferential edge for snapping onto a circumferential bulge on the proximal end of the trocar sleeve. In the case of this embodiment, the cover plate can be designed as a disposable part, while the trocar and/or the trocar sleeve can be a reusable component.

The cover plate can be closed originally and cut into and opened by the penetrating trocar. In accordance with another embodiment, the cover plate has from the beginning at least one slit for the piercing of the trocar. A controlled opening of the cover plate is hereby enabled, which favors a repeated sealing after removal of the trocar. The number of slits in the covering wall preferably matches the number of edges of the multi-edge tip of the trocar, wherein the slits are aligned starting from a central cutting point according to the alignment of the multi-edge tip. The trocar can be aligned with the multi-edge tip on the slit, whereby a particularly controlled opening of the valve is achieved.

In accordance with one embodiment, the valve has at least one weakening line for at least partial ripping of the valve. After at least partial ripping, the valve can be more easily removed from the trocar sleeve. The weakening line is for example a line, along which the wall thickness of the valve is reduced or a perforation line. In accordance with a further embodiment, the valve has a pull tab on the edge side. The pull tab facilitates the initial tearing of the valve along the weakening line. In accordance with a further embodiment, the weakening line runs in the radial direction and/or in the circumferential direction of the cover plate. When the weakening line extends in the radial direction, the cover plate can be separated into segments. If the cover plate has a circumferential edge, the weakening line preferably extends in the axial direction over the circumferential edge so that the cover plate can be ripped open starting from the edge. Especially in the case of a cover plate with circumferential edge, the weakening line can advantageously progress in the circumferential direction of the cover plate in order to enable at least partial pulling off of the edge along the weakening line.

The valve or respectively the cover plate is made for example of silicon or rubber or TPE (thermoplastic elastomer).

The kit according to the invention for providing an artificial stomach entrance comprises a trocar with a trocar sleeve for penetrating the abdominal wall into the stomach and a PEG tube insertable into it after removal of the trocar from the trocar sleeve, wherein the trocar sleeve has a marking on the perimeter at a distance from the distal end for displaying whether the trocar sleeve is pushed deeply enough into the abdominal wall and stomach.

In the case of the kit according to the invention, the trocar with the trocar sleeve is pushed into the abdominal wall and stomach wall in the already described manner. In the case of conventional kits, the extent of the insertion is left to the experience of the operator. This could lead to an insufficient penetration of the trocar and trocar sleeve, with the risk that the stomach wall slides off the trocar sleeve and the PEG tube is incorrectly placed. With the kit according to the invention, a correct penetration depth of the trocar sleeve into the abdominal wall and stomach wall is guaranteed with the help of the marking. The distance between the marking and the distal end of the trocar sleeve is based on experience. If necessary, different markings can be applied, which specify an optimal position depending on age, sex, weight, etc.

In accordance with one embodiment, the marking is arranged at a distance of 2 to 5 cm from the distal end of the trocar sleeve. This area is based on experience. It is preferably arranged at a distance of approx. 3 cm from the distal end of the trocar sleeve. The marking can be designed differently, for example as a groove or small embossment on the perimeter of the trocar sleeve. In accordance with one embodiment, it is a line, which is for example imprinted or painted on.

A kit for providing an artificial stomach entrance comprises a trocar with a trocar sleeve for penetrating the abdominal wall into the stomach and a PEG tube insertable into it after removal of the trocar from the trocar sleeve made of a flexible material, which has a distal extension insertable into the stomach, which has at least one opening, and a tubular area connecting proximally to the extension, wherein a feeding channel progresses from a proximal feeding opening of the tubular area through the tubular area up to the at least one opening of the extension, and a rod with a distal end and a handle on the proximal end, which can be pushed into the feeding channel with the distal end.

In the case of the kit according to the invention, the trocar with the trocar sleeve is placed in the described manner and the trocar is removed from the trocar sleeve. For placing the PEG tube, the rod is inserted into it and the flexible PEG tube is hereby stabilized. The insertion of the PEG tube into the trocar sleeve and its correct placement are hereby favored. The distal end of the rod is preferably blunt.

The rod can be designed or respectively applied such that it serves exclusively for the stabilization of the moldable PEG tube during insertion into the trocar sleeve. But it can also be designed or respectively applied such that it stretches the PEG tube during insertion. In particular for this purpose, the PEG tube is made of an elastic material in accordance with one embodiment. Furthermore, in accordance with one embodiment, the rod with a distal end can be pushed against an abutment on the distal end of the PEG tube. The stretching of the PEG tube is associated with a reduction in its outer diameter and this enables the use of a trocar and a trocar sleeve with a smaller cross-section. The traumatization of the patient during the placement of the artificial stomach entrance is hereby reduced.

In accordance with one embodiment, the abutment is a bottom on the distal end of the PEG tube or a step in the feeding channel, onto which the rod with the distal end can be placed. The distal end of the rod is preferably blunt. If the abutment is a bottom of the PEG tube, at least one opening of the feeding channel can be designed proximal to the bottom. The step is preferably a circumferential step. It can be designed in particular as a ring, which is embedded in the feeding channel. The step is preferably arranged on the distal end of the feeding channel. The abutment can also be a plug, which entirely or partially closes the feeding channel and dissolves in the patient's stomach. In accordance with another embodiment, the abutment is at least one eyelet on the distal end of the PEG tube, into which the rod with a projection on its distal end can be inserted. The eyelet can be designed for example on a flexible strap, which is arranged on the distal end of the PEG tube. The PEG tube preferably has two diametrically opposed eyelets on its distal end. Accordingly, the rod is preferably provided with two fork-like projections on its distal end. With its projections, the rod can engage in the eyelets of the straps, which are pivoted inward over the opening of the feeding channel. After pulling the rod out of the eyelets, they can fold outward and release the opening of the feeding channel.

In accordance with one embodiment, the PEG tube has an extension on the proximal end. The extension facilitates the stretching of the PEG tube with the help of the rod, in that the operator manually pushes the extension and the handle together.

In accordance with a further embodiment, the PEG tube has a seal on the proximal end. The seal serves to close the feeding channel when no food is fed. Furthermore, the seal can form an extension on the proximal end, which facilitates the placement of the PEG tube in the stretched state with the help of the rod.

In accordance with a further embodiment, the PEG tube on the distal end has a balloon and a fill channel connected with it and extended along the tubular area up to the proximal end with a seal on a proximal fill opening, wherein a fluid for pumping up to the balloon can be filled through the fill channel. The balloon forms the distal extension. Through the filling of a fluid, i.e. a liquid or a gas, it can be expanded after the placement of the PEG tube, in order to hold it on the inside of the stomach wall.

The kit according to the invention for providing an artificial stomach entrance comprises a trocar with a trocar sleeve for penetrating the abdominal wall into the stomach and a PEG tube made of an elastically deformable material with a distal extension insertable into the stomach in the form of a balloon, which has at least one opening and a proximally connecting tubular area, wherein a feeding channel extends from a proximal feeding opening of the tubular area through the tubular area up to the at least one opening and the balloon is connected with a fill channel, which extends along the tubular area up to the proximal end and has a fill opening with a seal, wherein the seal has a removable closing element blocking the cross-section of the fill opening, the closing element and the PEG tube on the proximal end have means for locking the closing element on the PEG tube and the closing element has a means for the use of a key for the purpose of cancelling the lock of the closing element with the PEG tube.

In the case of the kit according to the invention, the trocar with the trocar sleeve and the PEG tube is placed in the already described manner. A fluid is filled through the fill opening in order to anchor the PEG tube on the inner wall of the abdominal wall. Finally, the fill opening is blocked by the closing element, which is locked with the PEG tube. For unlocking the closing element, the use of a key is required, which must be applied to the means for applying the closing element. It can hereby be a conventional key, which can be used in a lock integrated with the closing element and works together with the means for locking. In accordance with a preferred embodiment, the means for applying a key are a tool application point for applying a tool and the key is formed by a tool. The tool is preferably a special tool. The necessity of the use of a key prevents the fluid from accidentally getting let out of the balloon and the PEG tube slipping out of the stomach. As a result, with the kit, the correct positioning of the PEG tube is ensured by the closing element. As a result, dangerous situations cannot arise, such as the feeding of liquid food between the abdominal wall and the stomach wall, which has already led to instances of death.

In accordance with one embodiment, the locking element is a threaded pin, which can be screwed into an internal thread of the fill opening.

In accordance with another embodiment, the tool application point is a tool application point for a screw wrench, a screw driver, an internal multi-edge wrench or another turning tool. The tool application point is for example a slit, a cross recess, a torx wrench or Allen key.

In accordance with another embodiment, the tool application point has at least one slit, internal multi tooth or internal multi edge.

A kit according to the invention for providing an artificial stomach entrance comprises a trocar with a trocar sleeve for penetrating the abdominal wall into the stomach and a PEG tube insertable into it after removal of the trocar from the trocar sleeve with a distal extension insertable into the stomach, which has distally at least one opening, and a proximally connecting tubular area, wherein a feeding channel extends from a proximal feeding opening of the tubular area through the tubular area up to the at least one opening, wherein the PEG tube has a marking at a distance from the distal extension and/or the proximal end, for displaying whether the PEG tube is pushed deep enough into the trocar sleeve.

The marking at a distance from the distal extension and/or from the proximal end facilitates the correct insertion of the PEG tube into the trocar sleeve. In particular, upon arrangement of the marking at a small distance from the distal extension, it can be observed using a gastroscope that the PEG tube is pushed in deep enough. Without a marking, it may be very difficult to identify the distal extension, in particular if it is designed as a balloon, which lies tightly against the PEG tube. This marking can be arranged for example approx. 1 to 2 cm proximal from the distal extension. If the trocar sleeve is correctly attached, it is generally also possible to identify the correct positioning of the PEG tube outside with the help of the marking, which is arranged at a small distance from the proximal end.

In accordance with a preferred embodiment, the PEG tube has a distal extension in the form of a balloon.

A kit according to the invention for providing an artificial stomach entrance comprises a trocar with a trocar sleeve for penetrating the abdominal wall into the stomach and a PEG tube insertable into it after removal of the trocar from the trocar sleeve with a distal extension insertable into the stomach in the form of an elastic balloon with an insertion sleeve surrounding the balloon, in which the balloon is arranged pressed together radially elastically, wherein the insertion sleeve with the PEG tube arranged in it is insertable into the trocar sleeve and the PEG tube with the balloon can be pushed out of the insertion sleeve and out of the trocar sleeve in the distal direction so that the balloon expands.

The insertion sleeve serves to press together the elastic balloon in the radial direction so that the PEG tube in the insertion sleeve can be pushed into the trocar sleeve. Moreover, the insertion sleeve protects the balloon during movement in the trocar sleeve. When the PEG tube with the balloon is pushed out of the insertion sleeve, the balloon expands. If this occurs in the trocar sleeve, it lies on the inner wall of the trocar sleeve. The PEG tube can then be pushed in further until the balloon fully expands in the stomach. But it is also possible to push the PEG tube into the stomach within the insertion sleeve with the balloon and to only then pull back the insertion sleeve so that the balloon fully expands in the stomach immediately. The balloon can be made entirely of an elastic material. But it is also possible to design the PEG tube with a balloon, which has a flexible or elastic wall and is permanently filled with an elastic medium. The elastic material, from which the balloon is formed or with which the balloon is filled, can be for example an elastic foam.

In the case of this embodiment, the PEG tube can be preinstalled in the insertion sleeve and inserted into the trocar sleeve. The PEG tube, above all the balloon, can thus have a reduced outer diameter and the balloon with the PEG tube is additionally protected during insertion by the insertion sleeve.

In accordance with the embodiments of the balloon made of an elastic material, the PEG tube has a very good, secure hold on the stomach wall. This is very good for long-term feeding. But, this PEG tube can only be removed via the stomach and the esophagus.

In the case of embodiments of the PEG tube made of an elastic material or respectively with a filling made of an elastic material, an adhesive or another stabilization means can be introduced to the balloon through a fill channel. The stabilizing material serves to reduce the elasticity of the balloon. This can be a hardening adhesive, for example a fibrin glue.

In accordance with one embodiment, the balloon on the PEG tube can be filled with a substance, the consistency of which is changeable, for example through factors such as temperature or the like. Thus, the substance can be added to the balloon at a very low viscosity and manipulated via the feeding channel of the PEG tube such that the substance in the balloon becomes more viscous and thus forms a greater resistance when the balloon is pulled against the stomach wall. During the pulling out of the PEG tube, this substance can be made less viscous again so that it can be suctioned out of the balloon and the PEG tube can be removed.

In accordance with a preferred embodiment, the insertion sleeve is a sleeve dividable in the longitudinal direction in order to remove the insertion sleeve from the trocar sleeve after the pushing out of the balloon. The divisibility of the insertion sleeve enables in particular the pulling out of the insertion sleeve when the PEG tube has an extension on the proximal end.

In accordance with a preferred embodiment, the insertion sleeve is made of a plastic.

In accordance with another preferred embodiment, the insertion sleeve has a handle on the proximal end. The handle facilitates the pushing in of the insertion sleeve in the trocar sleeve.

The following advantageous embodiments apply to all previously described kits.

In the case of a kit with a PEG tube, which has a balloon, which is fillable via a fill channel, a liquid medium, which can be brought into a solid or elastic state in the balloon via body heat or another activation (e.g. through radiation), can be introduced into the fill channel according to one embodiment. This makes it possible to fix the PEG tube securely on the inner wall of the stomach. In order to remove the PEG tube, a medium is preferably used that can be returned to its original liquid state, for example through targeted cooling or deactivation by means of another radiation.

In accordance with one embodiment, the kit comprises a hardenable medium for filling a balloon of the PEG tube. The medium is preferably liquid during the filling of the balloon and is then hardened through activation in the balloon, e.g. through the effect of radiation or heat. It also preferably has the property to be able to be returned back to the liquid state from the hardened state, e.g. through heat removal or supply of another radiation. This serves to empty the balloon and to remove the PEG tube.

In accordance with another embodiment, the trocar sleeve can be broken down into at least two halves in the longitudinal direction in order to remove the trocar sleeve from the penetration channel in the abdominal wall and stomach after the placement of the PEG tube. A simple pulling out of the trocar sleeve from the PEG tube is not possible in particular when the PEG tube has an extension on the proximal end, which serves for example as a handle during the placement of the PEG tube or as a seal for the feeding channel or for the fill channel.

In accordance with a preferred embodiment, the trocar sleeve and/or the trocar is made of plastic. The plastic is e.g. PEK (polyetherketone).

In accordance with a further embodiment, the trocar sleeve and/or the trocar is made of metal, in particular stainless steel or titanium.

In accordance with a further embodiment, the PEG tube is made of plastic and/or silicon and/or PUR. Combinations of the names materials are also possible. For example, the balloon is made of PUR and the rest of the PEG tube is made of another plastic or silicon.

In accordance with a further embodiment, the trocar has a guide channel passing through in the longitudinal direction for a guide wire. This enables the placement of the PEG tube after lying of a guide wire under observation e.g. via ultrasound without gastroscopy. After lying of the guide wire, the trocar with the trocar sleeve is pushed in through the guide shaft and then the PEG tube is placed. According to the invention, this trocar can also be used with conventional trocar sleeves and/or PEG tubes.

With the help of the kit according to the invention, an artificial stomach entrance is established according to the following procedure:
1. A gastroscope is first inserted into the stomach via the mouth through the esophagus and air is blown into the stomach via the gastroscope in order to stretch it. The puncture point is determined and marked through illumination with the gastroscope from the stomach through the abdominal wall (diaphanoscopy) and through pressure with the finger on the abdominal wall.
2. The puncture area is anaesthetized locally with an injection.
3. A small incision is now made at the puncture point with a scalpel.
4. The trocar is now inserted into the stomach at the puncture point through the abdominal wall with the trocar sleeve designed as a splitting sleeve. This puncture is observed from inside with the gastroscope.
5. The trocar is removed from the splitting sleeve. The splitting sleeve has a seal on the proximal end so that the air cannot escape from the stomach.
6. Next, the PEG tube is placed on the rod serving for insertion and is inserted into the stomach through the splitting sleeve.
7. Then the balloon on the distal end of the PEG tube is filled with liquid from the outside via a valve, in order to be able to pull the stomach onto the abdominal wall and in order to avoid a pulling out of the PEG tube. This procedure is monitored with the gastroscope lying in the stomach.
8. The splitting sleeve is now pulled out of the abdominal wall, then broken open and removed.
9. The rod is now pulled out of the PEG tube.
10. From the outside, a holding plate is fastened on the abdominal wall for the PEG tube.
11. There are now two connections to the PEG tube: one for the food and one with a valve, via which the balloon was filled. In order to not mix up the two connections, a protective cap is now placed on the connection with the access to the valve, which can only be removed with a special instrument.

The PEG tube can be designed in particular like the implant for percutaneous food supply in accordance with EP 0 667 763 B1. The implants specified in the exemplary embodiments are referenced in particular. These embodiments are included in the present application through reference.

An exemplary embodiment of the invention is explained below based on the attached drawings. The drawings shown in:

### BRIEF DESCRIPTION OF THE SEVERAL VIEWS OF THE DRAWINGS

FIG. 1 through 3 a.) through e.) and FIG. 4a.) through d.) four embodiments of trocars with particularly sharp trocar tips in a side view (FIG. 1a through 4a), front view (FIG. 1b through 4b), an enlarged partial view from the front (FIG. 1c through 4c), a perspective view (FIG. 1d through 4d) and in an enlarged partial view from the side (FIG. 1e through 3e);

FIG. 5 a.) through c.) a trocar sleeve with valve on the proximal end (FIG. 5a) and the removed valve (FIG. 5b) each in a side view and the removed valve in the top view (FIG. 5c);

FIGS. 6 a.) and b.) a PEG tube with balloon on the distal end and closing elements on the proximal end in the side view (FIG. 6a) and a rod for the stabilization of the PEG tube in a side view (FIG. 6b);

FIGS. 7 a.) and b.) a PEG tube with balloon on the distal end and closing elements on the proximal end in the side view (FIG. 7a) and a rod for the stretching of the PEG tube in a side view (FIG. 7b);

FIG. 8 a.) through c.) an alternative trocar sleeve with valve on the proximal end (FIG. 8a) and the removed valve (FIG. 8b) each in a side view and the removed valve in the top view (FIG. 8c); FIGS. 9a.) and b.) an alternative PEG tube with balloon on the distal end and closing elements on the proximal end in the side view (FIG. 9a) and a rod for the stabilization of the PEG tube in a side view (FIG. 9 b);

FIGS. 10 a) and b) alternative PEG tubes with abutments on the distal end in the form of eyelets, with a rod for stretching with fork-like projections on the distal end in a longitudinal cut (FIG. 10 a) and in the form of an annular stop for placement of a blunt distal end of the rod in a longitudinal cut (FIG. 10 b);

FIG. 11 a trocar sleeve with an inserted insertion sleeve with a PEG tube arranged in it with an elastic balloon in a longitudinal cut.

### DETAILED DESCRIPTION OF THE INVENTION

While this invention may be embodied in many different forms, there are described in detail herein a specific preferred embodiment of the invention. This description is an exemplification of the principles of the invention and is not intended to limit the invention to the particular embodiment illustrated

In the case of the following explanation of different exemplary embodiments, mainly corresponding elements are provided with the same reference numbers.

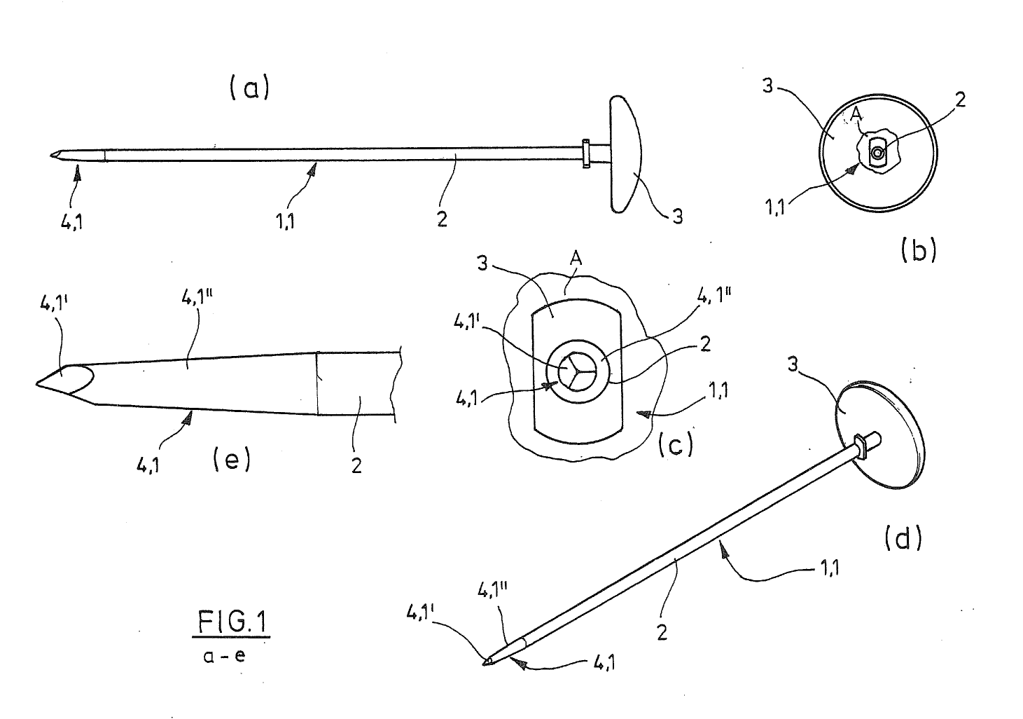

FIG. 1 through 4 show different trocars 1.1, 1.2, 1.3, 1.4, each of which has a rod section 2 and a knob-like handle 3 on the proximal end of the rod section.

In accordance with FIG. 1, the trocar 1.1 on the distal end of the rod section has a trocar tip 4.1, which is designed in the front with 4.1′ as a triangular tip and in the back with 4.1″ as a conical tip. The polished angle in the area of 4.1′ is 20° or higher. With the area 4.1′, a hole is bored. The half conical angle in the area 4.1″ is 3°. The half conical angle is significant for the gradual piercing and penetration of the abdominal wall and stomach wall.

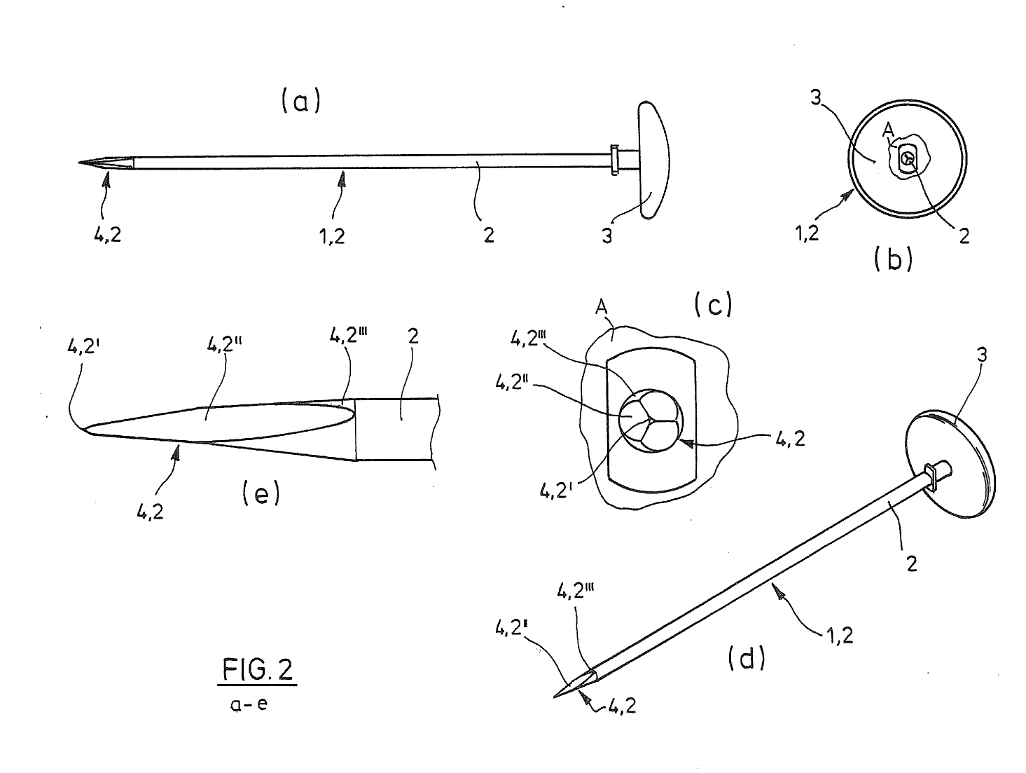

In accordance with FIG. 2, the trocar 1.2 has a triangular tip 4.2 in the front. The triangular tip 4.2 has a short sharpening 4.2′ with a conical angle of approx. 40°. The area 4.2′ serves for the boring of a hole. Then comes a longer area 4.2″, which is polished. The polished angle is approx. 6.5°. This polished angle is significant for the gradual piercing and penetration of the abdominal wall and stomach wall. A part of the area 4.2″ overlaps a conical area 4.2′″. The half conical angle is 3°. A gradual piercing and penetrating is also favored from this area 4.2′″.

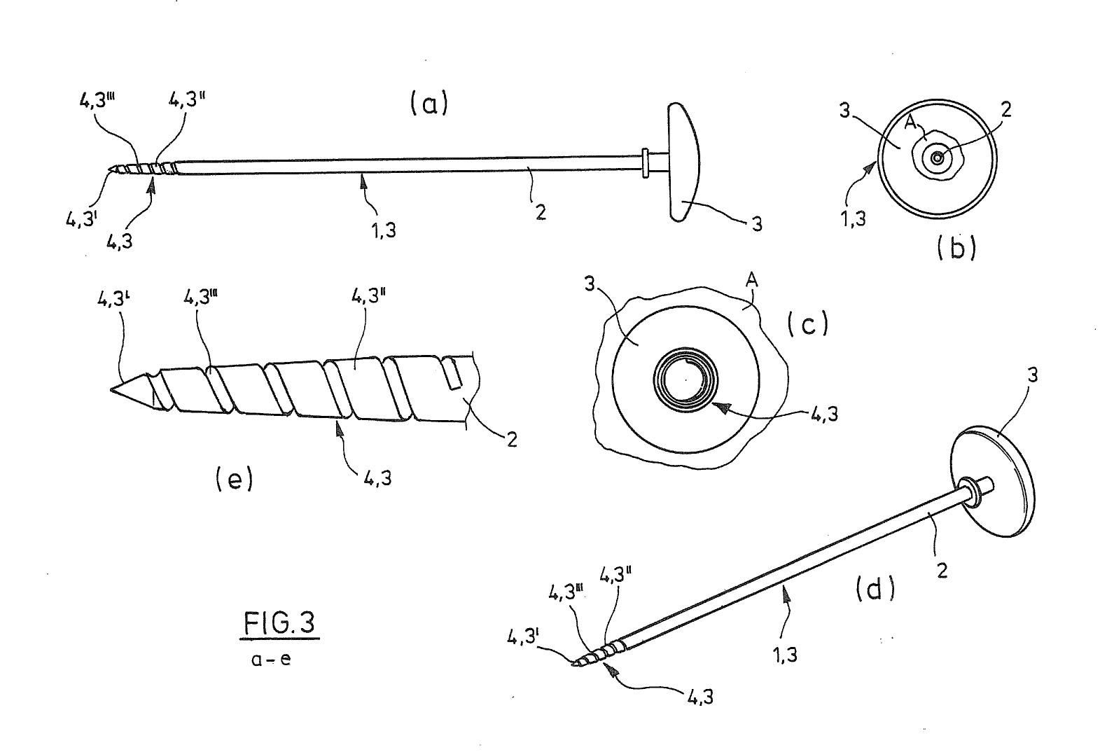

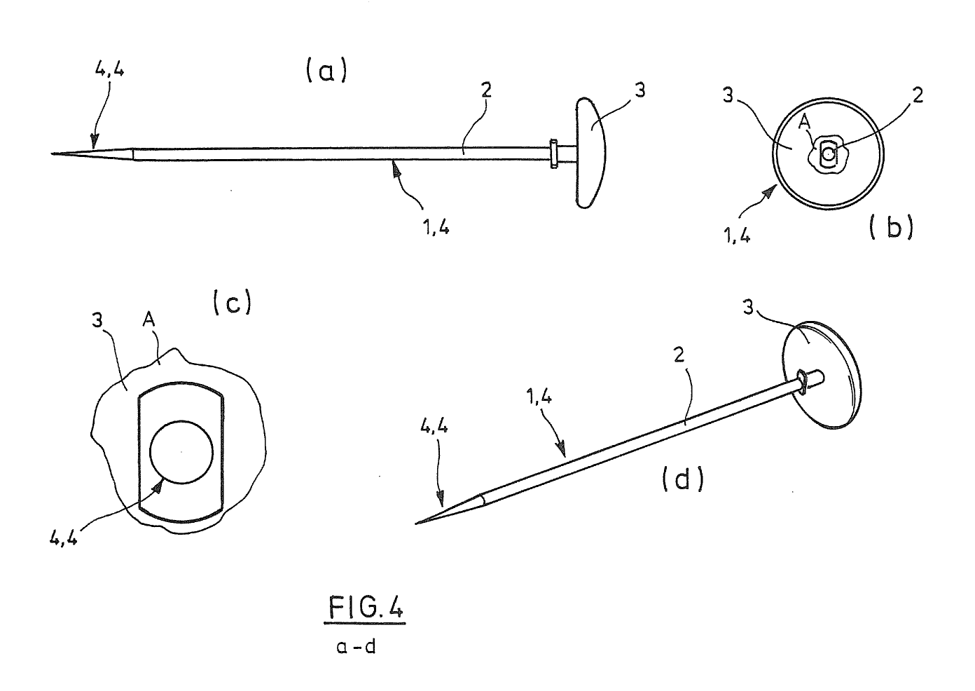

In accordance with FIG. 3, the trocar 1.3 has a conical tip 4.3 in the front. The conical tip 4.3 is provided on the front, distal end with a conical sharpening 4.3′, the conical angle of which is approximately 40°. This area 4.3′ serves for the boring of a hole. A long, conical area 4.3″ connects to it, which has a half conical angle of 3°. The area 4.3″ is significant for the gradual piercing and penetration of the abdominal wall and stomach wall. The area 4.3″ is provided with an external thread 4.3′″. This supports a gradual piercing through the screwing in of the trocar 1.3.

In accordance with FIG. 4, the trocar 1.4 has a conical tip 4.4 with a half conical angle of approximately 6.5°. The conical tip 4.4 is designed exclusively as a smooth conical tip, i.e. it is not subdivided into areas. It favors a gradual piercing and penetration of the abdominal wall and stomach wall.

The trocar tips 4.1, 4.2, 4.3, 4.4 all have a polished angle or respectively a half conical angle of max. 20° in the areas significant for the piercing and penetration of the abdominal wall and stomach wall. The polished angle is the angle between the lateral surfaces of a triangular tip and the middle axis of the trocar. The conical angle is the opening angle of the cone.

The length of the trocar tips 4.1 through 4.4 is 19 mm in the example. It can vary depending on the diameter of the trocar 1.1 through 1.4. The length can be e.g. only approximately 5 mm in a trocar with a small diameter and 40 mm in a trocar with a large diameter. It is preferably limited to 20 mm. The entire length in the axial direction of the trocar tip 4.1 is approx. 19 mm.

Furthermore, the trocars 1.1, 1.2, 1.3, 1.4 each have a diameter of 4.35 mm in the area of the rod section 2. The diameter of the trocar varies preferably in the range of 4 mm to 8 mm.

Based on the novel, sharp embodiment of the trocar tips 4.1 through 4.4, the named trocars are gradually insertable into the abdominal wall and the stomach of a patient.

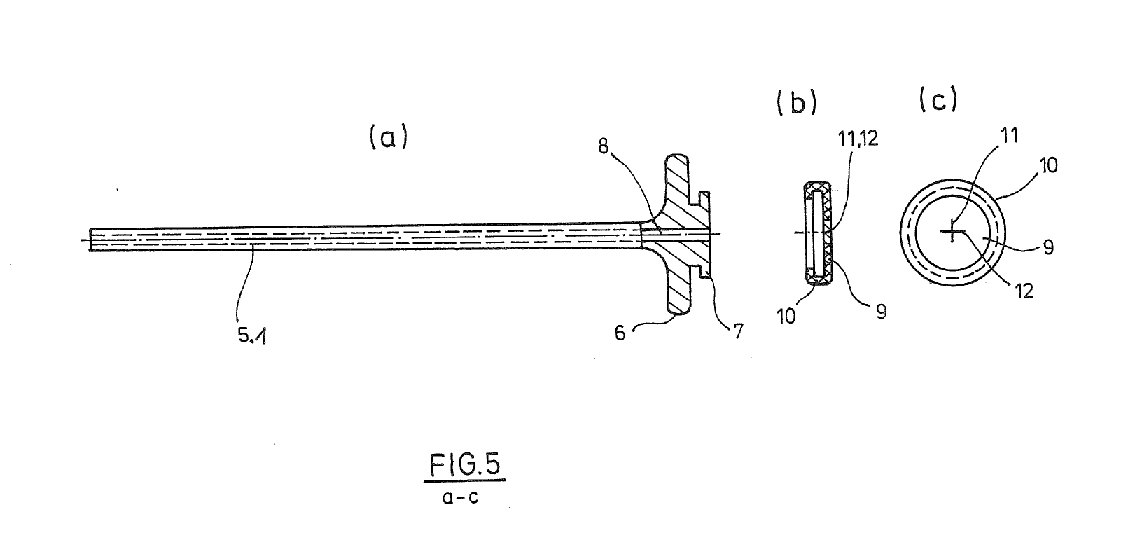

In accordance with FIG. 5, a trocar sleeve 5.1 designed as a splitting cannula has a knob-like handle 6 on the proximal end, which has a circumferential flange 7 on the outer front side, which is hook-shaped in the longitudinal cut.

The trocar sleeve 5.1 has a passage channel 8 for inserting a trocar and a PEG tube.

A cover plate 9 made of an elastic material is placed on the flange 7, which has an outwardly bent, circumferential edge 10. With the edge 10, the cover plate 9 is snapped onto the flange 7.

The cover plate has two crossing slits 11, 12 in the center, which form a slit valve.

Three slits can also be present, which intersect at a middle point.

The trocar 1 is insertable into the passage channel 8 of the trocar 5 through the slit valve. After removing the trocar 1 from the cover plate 9, the slits close and excess pressure cannot escape from the passage channel 8.

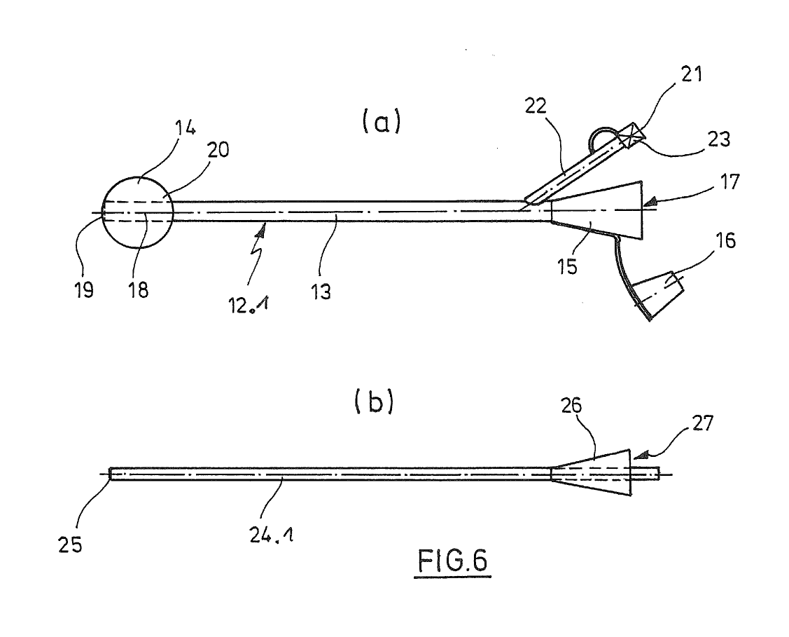

In accordance with FIG. 6 a), a PEG tube 12.1 has a tubular area 13 with a balloon 14 on the distal end and an extension 15 on the proximal end, which is assigned to a sealing plug 16, which is insertable into an infeed opening 17 within the extension. The sealing plug 16 is connected as one piece with the extension 15 surrounding the feeding opening 17 via a strap. A feeding channel 18 is connected with the feeding opening 17, which runs through the tubular area 13 and the balloon 14 and opens in an opening 19 on the distal end.

Furthermore, the PEG tube 12.1 has a fill channel 20, which extends starting from a fill opening 21 in a tube piece 22 protruding laterally from the proximal end of the tubular area 13 through the tubular area 13 up to channel 14. The fill opening 21 is closed by a valve 23, which is connected as one piece with the tube piece 22 via a strap.

The PEG tube 12.1 consists of an elastic, flexible material such as silicon.

Furthermore, the kit has a rod 24.1, which has a blunt, proximal end 25 and a handle 26 with a front-side pressing surface 27.

The rod 24.1 can be inserted for stabilization into the feeding channel 18 through the feeding opening 17. The rod 24.1 is then pulled out. In this arrangement, the PEG can be easily inserted into a trocar sleeve 5. After the placement of the PEG tube 12.1, the balloon 14 is blown up through filling of a fluid through the fill opening 21. The fill opening 21 is then closed by the seal 23.

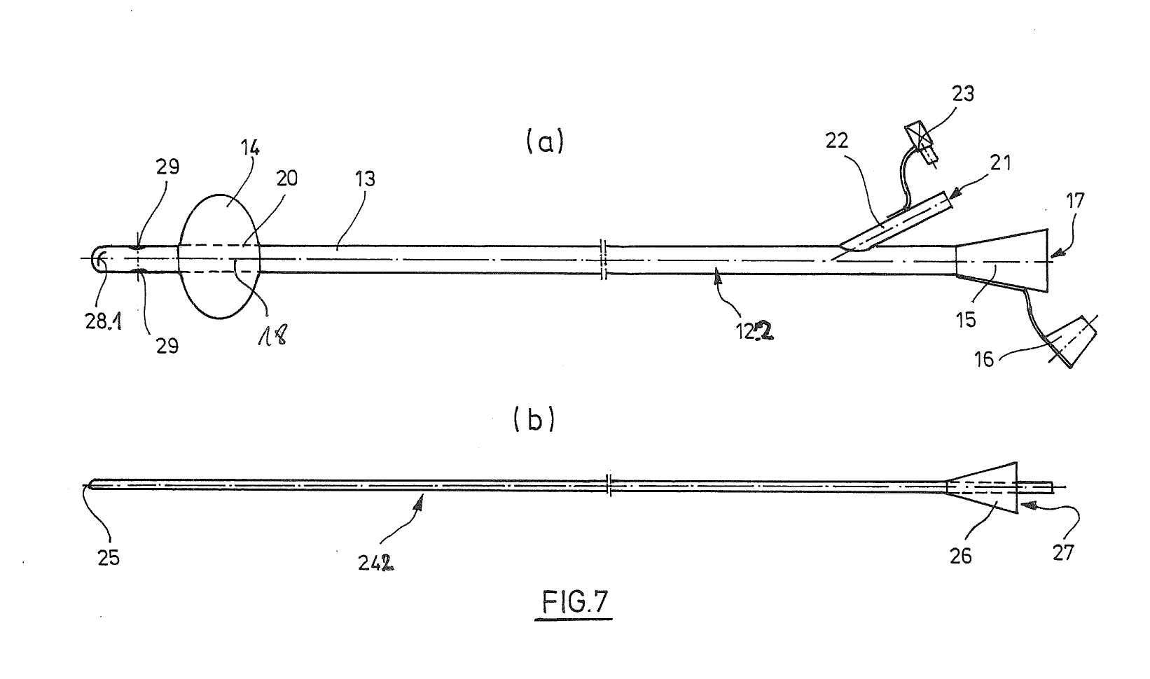

FIG. 7a) shows a PEG tube 12.2, which differs from that previously described in particular in its longer length. Furthermore, the balloon 14 does not sit directly on the distal end, but is rather arranged at a distance from it. The tubular area 13 is closed by a bottom on the distal end and there forms an abutment 28.1. Radial passage openings 29 that are connected with the feeding channel 18 are present next to the abutment.

In accordance with FIG. 7b), an expansion rod 24.2 is in particular longer compared to the expansion rod in FIG. 6b). The expansion rod 24.2 can be inserted through the infeed opening 17 into the PEG tube 12.2, until it presses against the abutment 28.1 with its blunt, distal end 25. Through the pressing together of the extension 15 and the handle 26, the PEG tube 12.2 can be stretched so that it can pass through a trocar sleeve 5, which has a particularly narrow lumen or respectively cross-section.

In order to avoid an unintentional collapse of the balloon 14 if the PEG tube 12.1 as per FIG. 6 a) or respectively 12.2 as per FIG. 7 a), the fill channel 20 can be filled with a flowing medium instead of a liquid (e.g. water), which hardens after the filling of the balloon. It can hereby be for example an adhesive, in particular an adhesive that is mixed together from several components during or before the filling of the balloon. The medium can be rigid or elastic after the hardening. It is preferably elastic in order to facilitate the removal of the PEG tube 12.1 or respectively 12.2. For this, the tubular area 13 outside the patient's body is cut off and the part remaining in the body is pulled out through the esophagus with the balloon 14 by means of forceps. Alternatively, the balloon 14 can be provided on the perimeter with an elastic material ring or another elastically deformable area, which is not filled with the hardening medium. This elastically deformable area facilitates the pulling of the distal section of the PEG tube 12.1 or respectively 12.2 out of the esophagus.

When using the hardening medium, it can be used as part of a kit.

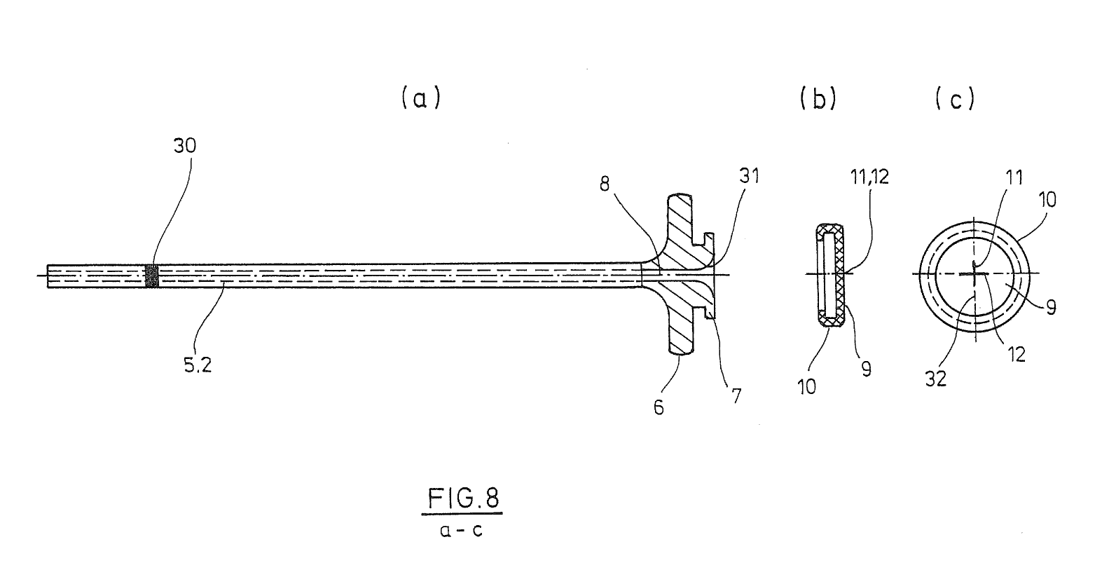

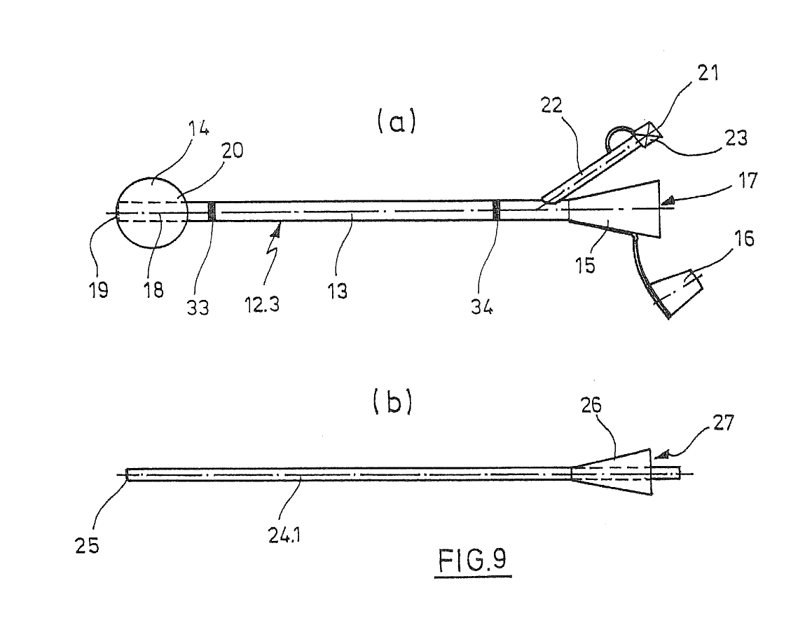

The trocar sleeve 5.2 from FIG. 8 differs from the trocar sleeve 5.1 in that it has a marking 30 in the form of an annularly circumferential line on the perimeter in the vicinity of the distal end. The marking 30 can be embedded into the material of the trocar 5.1 or can be an applied paint. Furthermore, the trocar sleeve 5.2 has a conically outwardly expanding insertion area 31 for a PEG tube on the proximal end. The insertion area 31 facilitates the pushing in of a PEG tube.

Another uniqueness is that the cover plate 9 is provided with a perforation line 32, which extends radially over the cover plate 9 axially over the perimeter of the edge 10. The cover plate 9 can be partially separated along the perforation line 32 in order to more easily remove it from the flange 7.

The PEG tube 12.3 from FIG. 9 differs from the PEG tube 12.1 from FIG. 6 in that the tubular area 13 is provided with a distal marking 33 and a proximal marking 34. These are designed respectively over the perimeter of the tubular area 12 as progressing, annular lines. They can be embedded into the material of the PEG tube 12.1 or painted on.

The distal marking 33 is arranged at a short distance of approx. 1 cm from the balloon 14. The proximal marking 34 is arranged at a comparatively short distance of approximately one or more centimeters from the extension 15. The operator can easily detect using the gastroscope when the marking 33 leaves the trocar sleeve. The PEG tube 12.3 is then pushed sufficiently far into the trocar sleeve. One can also detect the sufficient pushing of the PEG tube 12.3 into the trocar sleeve in that the marking 34 reaches the proximal end of the trocar sleeve. This suffices for orientation when the trocar sleeve is correctly inserted into the abdominal wall and stomach, which can be checked using the gastroscope.

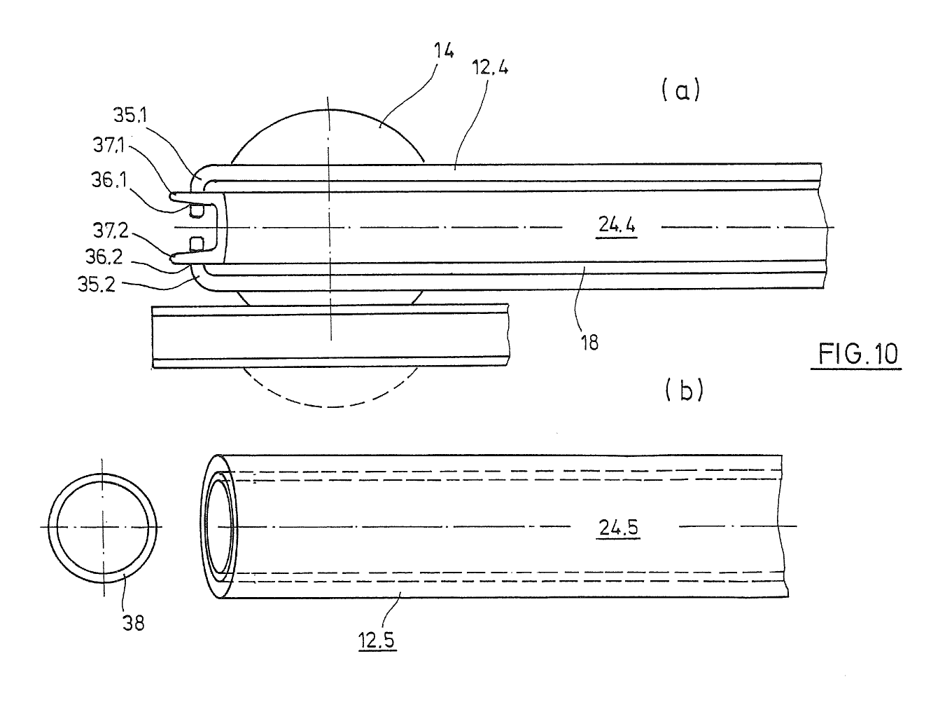

In accordance with FIG. 10 a), a PEG tube 12.4 has two flexible straps 35.1, 35.2 on the distal end, each of which have a hole 36.1, 36.2 and each of which form an eyelet. A rod 24.4 is provided with two fork-like projections 37.1, 37.2 on the distal end. The flexible straps 35.1, 35.2 are foldable into the distal opening of the feeding channel 18 so that the rod 24.4 is insertable into the holes 36.1, 36.2 with the projections 37.1, 37.2. In this manner, the expansion rod 24.3 is held distally and can be used for stretching the elastic PEG tube 12.4.

The embodiment from FIG. 10 b) differs from that described above in that the PEG tube 12.5 has an inserted ring 38 on the distal end, which is removed from the PEG tube 12.5 in the drawing for illustrative purposes. The ring 38 is fixed in the PEG tube 12.4 such that the rod 24.5 is pressable against the ring 39 with a blunt end, which forms an abutment. Thus the elastic PEG tube 12.5 is also stretchable by means of the expansion rod 24.5.

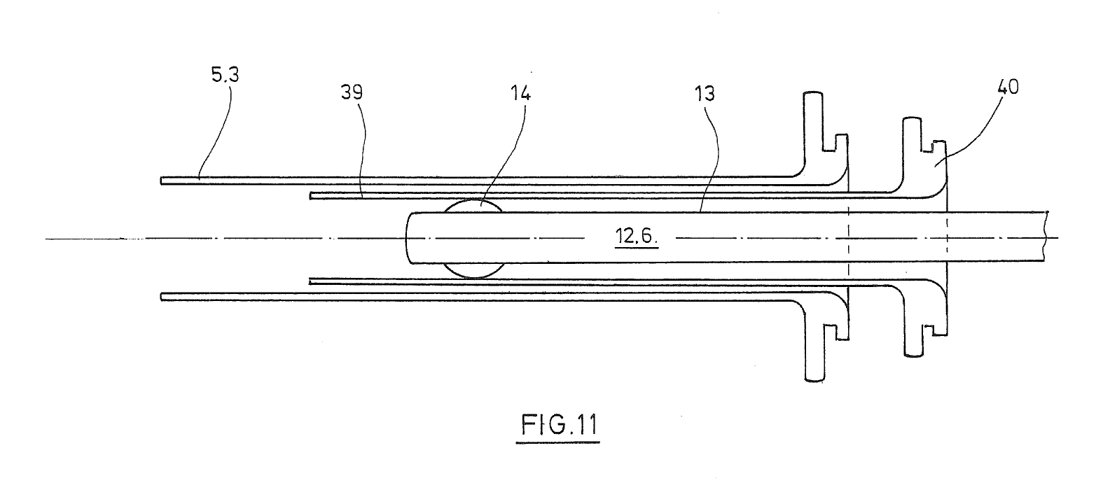

In the case of the kit in FIG. 11, the PEG tube 12.6 has a balloon 14 lying tightly on the tubular area. This is for example a balloon 14, which is made entirely of an elastic material or has a shell filled with an elastic material. Alternatively, it is a balloon 14, which consists of a flexible material of low stretchability. In the latter case, the balloon 14 can be fixed in the shown position on the tubular area 13 by means of a gel, which dissolves in the patient's stomach.

The balloon 14 is fixed in the state lying tightly against the tubular area 13 by an insertion sleeve 39. The insertion sleeve 39 has another handle 40 in the form of an area broadening outward radially on the proximal end.

In the insertion sleeve 39, the PEG tube 12.6 is pushed into a trocar sleeve 5.3. The balloon 14 is hereby fixed in the compressed position by the insertion sleeve 39. After removing the insertion sleeve 39, it can be divided in the longitudinal direction and removed, even if the PEG tube 12.6 is provided with a extension on the proximal end. If the balloon 14 is made of an elastic material or contains it, it expands radially so that it lies on the inner perimeter of the trocar sleeve 5.3. In the case of embodiment of the balloon 14 made of a flexible, preferably non-stretchable material, the balloon 14 continues to lie on the PEG tube 12.6.

The PEG tube 12.6 can then be pushed into the trocar sleeve 5.3 until the balloon exits it from the distal ends. An elastic balloon then expands automatically so that it can be supported on the stomach wall. A balloon 14 made of a flexible material is filled with a fluid through a fill channel of the PEG tube 12.6 so that it is blown up and can be supported on the stomach wall.

This completes the description of the preferred and alternate embodiments of the invention. Those skilled in the art may recognize other equivalents to the specific embodiment described herein which equivalents are intended to be encompassed by the claims attached hereto.

1-57. (canceled)

58. A Kit for providing an artificial stomach entrance, comprising a trocar (1) with a trocar sleeve (5) for penetrating the abdominal wall into the stomach and a PEG tube (12) with a distal extension insertable into the stomach in the form of a balloon (14) made of a flexible material that is not stretchable or if need has only slight stretchability, which is less stretchable than silicon, which has at least one opening (19) distally and a proximally connecting tubular area (13), wherein a feeding channel (18) extends from a proximal feeding opening of the tubular area through the tubular area up to the at least one opening (19) and the balloon is connected with a fill channel, which extends along the tubular area up to the proximal end and has a fill opening with a seal (23).

59. The kit according to claim 58, in which the balloon is made of PUR.

60. A Kit providing an artificial stomach entrance, comprising a trocar (1) with trocar sleeve (5) for penetration through the abdominal wall into the stomach and a PEG tube (12) insertable into it after removal of the trocar (1) from the trocar sleeve (5), wherein the trocar has a trocar tip (4), which is a conical tip (4″), in which the half conical angle is 5° to 12° and the trocar (1) has a diameter of 4 to 8 mm.

61. The kit according to claim 60, in which the trocar tip (4) has a length of 8 mm to 22 mm and/or in which the trocar tip (4) is provided with an external thread.

62. A Kit for providing an artificial stomach entrance, comprising a trocar (1) with a trocar sleeve (5) for penetrating the abdominal wall into the stomach and a PEG tube (12) insertable into it after removal of the trocar (1) from the trocar sleeve (5), wherein the trocar sleeve (5) has a valve (9) proximally, through which the trocar (1) can be inserted into the trocar sleeve (5) and which seals airtight after removal of the trocar (1) from the trocar sleeve (5), wherein the valve (9) has at least one weakening line (32) for at least partial ripping of the valve (9), the ripping of which facilitates the removal of the valve (9) from the trocar sleeve (5).

63. The kit according to claim 62, in which the trocar sleeve (5) and the valve (9) have holding means for releasably holding the valve (9) on the trocar sleeve (5) and/or in which the valve (9) and the trocar sleeve (5) have snapping means for snapping the valve (9) onto the trocar sleeve (5) and/or in which the cover plate (9) has an outwardly bent, circumferential edge (10) and the trocar sleeve (5) a circumferential flange (7) on the proximal end for snapping the cover plate (9) to the circumferential edge (10) and/or in which the valve comprises a cover plate (9) made of an elastic material or a material with a memory effect, which is affixed to the trocar sleeve (5) on the edge and blocks the passage channel (8) of the trocar sleeve (5) and through which the trocar (1) is piercable for insertion into the passage channel (8), wherein the penetration point (11, 12) in the cover plate (9) closes after removal of the trocar due to the elastic reset forces or the memory effect.

64. The kit according to claim 62, in which the valve (9) has at least one slit (10, 11) for inserting the trocar (1) and/or in which the number of slits (10, 11) in the cover plate (9) matches the number of edges of the multi-edge tip (4) of the trocar (1), wherein the slits are aligned starting from a central cutting point according to the alignment of the edges of the multi-edge tip of the trocar (1).

65. The kit according to claim 62, in which the valve has a pull tab on the edge side and/or in which the weakening line runs in the radial direction and/or circumferential direction of the cover plate.

66. The kit for providing an artificial stomach entrance, comprising a trocar (1) with a trocar sleeve (5) for penetrating the abdominal wall into the stomach and a PEG tube (12) insertable into it after removal of the trocar (1) from the trocar sleeve (5), wherein the trocar sleeve (5) has a marking on the perimeter at a distance from the distal end for displaying whether the trocar sleeve (5) is pushed deep enough into the abdominal wall and stomach.

67. The kit according to claim 66, in which the marking is arranged at a distance of 2 to 5 cm from the distal end of the trocar sleeve and/or in which the marking is a line arranged on the perimeter of the trocar sleeve.

68. Kit for providing an artificial stomach entrance, comprising a trocar (1) with a trocar sleeve (5) for penetrating the abdominal wall into the stomach and a PEG tube (12) insertable into it after removal of the trocar from the trocar sleeve made of a flexible material, which has a distal extension (14) insertable into the stomach, which has at least one distal opening (19), and a tubular area (13) connecting proximally to the extension, wherein a feeding channel (18) progresses from a proximal feeding opening (17) of the tubular area (13) through the tubular area up to the at least one opening (19), and a rod (24) with a distal end (25) and a handle (26) on the proximal end, which can be pushed into the feeding channel (18) with the distal end (25).

69. The kit according to claim 68, in which the PEG tube (12) is made of an elastic material and/or in which the length of the rod exceeds the length of the PEG tube so that the PEG tube is stretchable by means of the inserted rod and/or in which the PEG tube (12) has an abutment (28) for the rod (24) on the distal end and/or in which the abutment is a bottom of the PEG tube (12) or a step in the feeding channel (18), onto which the rod with the distal end can be placed, or at least one eyelet on the distal end of the PEG tube (12), into which the rod with a projection on the distal end can be inserted, or a plug in the feeding channel (18) dissolving in the environment of the stomach.

70. The kit according to claim 68, in which the PEG tube (12) has a broadening (26) on the proximal end and/or in which the PEG tube (12) has a seal (16) on the proximal end.

71. The kit according to claim 68, in which the PEG tube (12) on the distal end (25) has a balloon (14) and a fill channel (20) connected with it and extended along the tubular area (13) up to the proximal end with a seal (23) on a proximal fill opening (21), wherein a fluid for pumping up the balloon (14) can be filled through the fill channel (10).

72. Kit for providing an artificial stomach entrance, comprising a trocar (1) with a trocar sleeve (5) for penetrating the abdominal wall into the stomach and a PEG tube (12) with a distal extension insertable into the stomach in the form of a balloon (14), which has at least one opening (19) and a proximally connecting tubular area (13), wherein a feeding channel (18) extends from a proximal feeding opening (17) of the tubular area (13) through the tubular area up to the at least one opening (19) and the balloon (14) is connected with a fill channel (20), which extends along the tubular area (13) up to the proximal end and has a fill opening (21) with a seal (23), wherein the seal (23) has a removable closing element blocking the cross-section of the fill opening (21), the closing element and the PEG tube on the proximal end have means for locking the closing element on the PEG tube and the closing element has a means for the use of a key for the purpose of cancelling the lock of the closing element with the PEG tube (12).

73. The kit according to claim 72, in which the closing element is a threaded pin, which is screwable into an internal thread in the fill opening (21) and/or in which the means for applying a key is a tool application point and the key is a tool that can be placed on the tool application point.

74. The kit according to claim 72, in which the balloon (14) and/or the PEG tube (12) are made of a flexible or elastically deformable material.

75. Kit for providing an artificial stomach entrance, comprising a trocar (1) with a trocar sleeve (5) for penetrating the abdominal wall into the stomach and a PEG tube (12) insertable into it after removal of the trocar from the trocar sleeve with a distal extension (14) insertable into the stomach, which has distally at least one opening (19), and a proximally connecting tubular area (13), wherein a feeding channel (18) extends from a proximal feeding opening of the tubular area through the tubular area up to the at least one opening (19), wherein the PEG tube (12) has a marking (33, 34) at a distance from the distal extension and/or the proximal end, for displaying whether the PEG tube (12) is pushed deep enough into the trocar sleeve.

76. The kit according to claim 75, in which the PEG tube (12) has a distal extension in the form of a balloon (14).

77. Kit for providing an artificial stomach entrance, comprising a trocar (1) with a trocar sleeve (5) for penetrating the abdominal wall into the stomach and a PEG tube (12) insertable into it after removal of the trocar from the trocar sleeve with a distal extension insertable into the stomach in the form of an elastic balloon (14) with an insertion sleeve (39) surrounding the balloon, in which the balloon (14) is arranged pressed together radially elastically, wherein the insertion sleeve (39) with the PEG tube (12) arranged in it is insertable into the trocar sleeve (5) and the PEG tube (12) with the balloon (14) can be pushed out of the insertion sleeve (39) and out of the trocar sleeve in the distal direction so that the balloon (14) expands.

78. The kit according to claim 77, in which the balloon (14) is filled with an elastic foam and/or in which the insertion sleeve (39) is a sleeve dividable in the longitudinal direction in order to remove the insertion sleeve (39) from the trocar sleeve (5) after the pushing out of the balloon (14).

79. A Trocar, which has a guide channel for a guide wire extended in its longitudinal direction.

80. The kit according to claim 58 with a trocar according to claim 77.
